(19) United States
(12) Patent Application Publication
(10) Pub. No.: US 2006/0210615 A1
(43) Pub. Date: Sep. 21, 2006

(54) Dermal or transdermal therapeutic system comprising an ormocer with barrier effect on a cover foil

(76) Inventors: Frank Theobald, Bad Breisid (DE); Notger Weber, Neuwied (DE); Simon Günter, Hillesheim (DE)

Correspondence Address: William F Lawrence;Frommer Lawrence & Haug, 745 Fifth Avenue, New York, NY, 10151, US

(21) Appl. No.: 10/567,077

(22) PCT Filed: Jul. 23, 2004
(86) PCT No.: PCT/EP04/08221

§ 371 Date: Feb. 3, 2006

(30) Foreign Application Priority Data
Aug. 7, 2003 (DE) 10336211.8
Jun. 11, 2004 (DE) 10204028415.6

(51) Int. Cl. A61K 9/70

(52) U.S. Cl. 424/449

(57) ABSTRACT

The invention relates to a dermal or transdermal therapeutic system comprising a cover foil with barrier effect against gas, odour and volatile light substances. Said invention is characterised in that the cover foil is provided with at least one substrate and with at least one layer consisting of an inorganic-organic hybrid polymer (Ormocer).

The present invention relates to a dermal or transdermal therapeutic system comprising a covering film having a barrier effect against gases, aromas and readily volatile substances, which system is characterized in that the covering film is composed of at least one supporting layer and a layer composed of an inorganic-organic hybrid polymer (ormocer).

Dermal therapeutic systems are systems which bring a pharmaceutical, topically acting substance into contact with the affected skin segment or the affected musculature which lies below it; all so-called medicinal plasters belong to this category. Transdermal therapeutic systems, on the other hand, convey the active compound through the skin into the blood circulation and consequently act systemically. Both systems are essentially composed of a covering film (backing layer), an active compound reservoir or an active compound-containing matrix and a protective film which can be peeled off (release liner). The covering film remains on the dermal or transdermal therapeutic system during the application as well in order, on the one hand, to mechanically protect the active compound-containing matrix or the active compound-containing reservoir and, on the other hand, either to protect the active compound from the influence of gases, e.g. oxygen and water vapor, or to prevent the partial evaporation of a readily volatile active compound.

Both the covering film, which serves as backing layer, and the peelable protective film are generally composed of organic polymers. Examples of suitable materials for these films are: low density polyethylene (LDPE) and high density polyethylene (HDPE), polypropylene (PP), polyamide (PA), polyvinyl chloride (PVC), polyvinyl ester, polyethylene terephthalate (PET), copolymers composed of at least two of these polymers, or mixtures of these polymers. Since the covering film remains on the skin during the use of the system, it should be as flexible and elastic as possible in order to ensure appropriate comfort when being worn by the patient. Inflexible films lead to the formation of folds and to the skin being cut into. LDPE and HDPE, in particular, form suitable flexible and elastic covering films. However, all the abovementioned polymers, and, in particular, the two polyethylenes, suffer from the disadvantage that, to differing extents, they constitute good storage systems for active compounds. As a result, there is the danger that, during storage and/or use, the active compound may migrate out of the reservoir or the active compound-containing matrix and, by way of the gas space, into the covering film until the saturation concentration is reached. This thereby minimizes the active compound which is available in the matrix or the reservoir for permeation through the skin and minimizes the release kinetics of the dermal or transdermal therapeutic system.

DE 199 22 368A1 describes polymer compositions composed of water-soluble polymers and bound silicon dioxide building blocks, which are linked by polymerization, as material for pharmaceutical supporting materials. However, these compositions do not correspond to that of the ormocers.

DE 195 19 593 describes a transdermal therapeutic system for the release of volatile active compounds, which system comprises a backing layer which is essentially impermeable for the active compound and which is composed of a thermoplastic organic polymer.

WO 95/07817 describes a multilayer film which is impermeable for oxygen and moisture and which is produced by extraction from nylon and/or copolymers composed of ethylene and vinyl alcohol together with a thermoplastic organic polymer.

DE 199 58 554 A discloses a transdermal therapeutic system which comprises a backing layer which is impermeable for active compounds, at least one polymer layer, having microreservoirs which are contained therein, and at least one active compound and a protective layer which is to be removed prior to use. The material of the backing layer is composed of films such as polyethylene, polypropylene or polyester or of laminates of different polymers.

Amberg-Schwab S. et al: “Inorganic-organic polymers as migration barriers against liquid and volatile compounds”, Journal of Sol-Gel Science and Technology 26, 699-703, 2003. The publication describes the investigation of different, photochemically curable hybrid polymers, which are synthesized on the basis of the sol-gel technique, as a novel coating material for use as migration barriers having good properties with regard to printability, abrasion resistance and antistatics for the packaging industry.

WO 02/34510 A1 discloses a flexible sterile-goods packaging material which is composed of a film or a film composite having barrier properties towards gases, water vapor and aromas and which comprises a supporting film and, arranged on this, a thin ceramic layer onto which a functional layer composed of an inorganic-organic hybrid polymer is in turn applied. The material is suitable for sterilizing at high temperatures.

Knittel D. et al: “Surface of Textiles and the Human Skin: 1. Surface Modification of Fibers as Therapeutic and Diagnostic Systems”, Exogenous Dermatology 2003; 2, 11-16. The publication describes a new method for improving the skin-facing surfaces of textiles with cyclodextrins by anchoring them on the textile surface. In addition it is proposed that the textiles be functionalized with active compound-containing, inorganic-organic hybrid systems which are to serve as depots for controlled release.

It has furthermore already been proposed to reduce or completely prevent the permeability of flexible or elastic polymer layers for gases or volatile active compounds by combining polymers with metal layers, e.g. by means of a coating with aluminum or else by means of coating with metal oxides. Unfortunately, however, such a combination markedly impairs the flexibility, but in particular the elasticity, of these systems.

The object of the present invention is therefore to develop dermal or transdermal therapeutic systems having covering films which possess good flexibility and, at the same time, an outstanding barrier effect with regard to readily volatile active compounds or active compounds having a high vapor pressure.

According to the invention, this object is achieved by using covering films which are composed of organic polymers which are coated with an inorganic-organic hybrid polymer, i.e. what is termed ormocer.

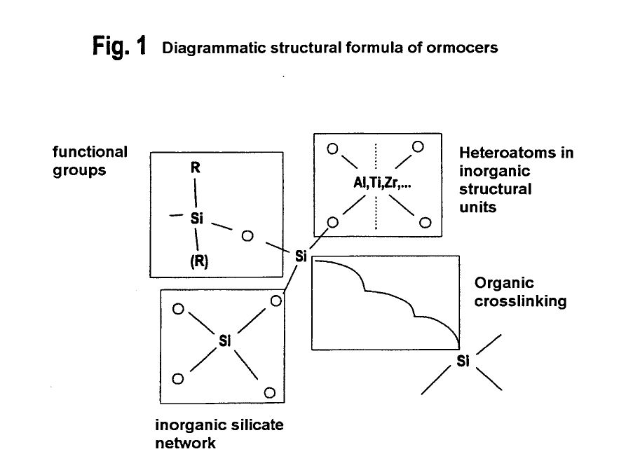

Inorganic-organic hybrid polymers, i.e. what are termed ormocers, have been known for some years. They are prepared in two steps in the following manner: initially, an inorganic network is synthesized by the controlled hydrolysis and condensation of organically modified silicon oxides, with a cocondensation with other metal alkoxides (Ti, Zr and Al alkoxides) also being possible. In a second step, the polymerizable groups which are linked to the inorganic network then react with each other as a consequence of thermal or UW treatment. Such a hybrid polymer then possesses the structural formula which is shown diagrammatically in FIG. 1.

The preparation and properties of ormocers have been described in the following publications: EP 0 358 011 A2; EP 0 373 451 A1; EP 0 610 831 A2; EP 0 644 908 B1; EP 0 792 846 A1; EP 0 934 989 A These publications are mentioned here expressly as being part of the disclosure.

Whereas it has not previously been possible to produce covering films, for dermal or transdermal systems, which possess good barrier properties and, at the same time, satisfactory elastic properties, it has now been found, surprisingly, that coating polymer films with ormocers, whose good barrier effect is already known, leads to covering films whose elasticity is either unimpaired or only insignificantly impaired. Ormocer layers having a thickness of between 1 μm and 10 μm are particularly suitable.

Both the covering film, which serves as backing layer, and the peelable protective film are generally preferably composed of organic polymers. Examples of suitable materials for these films are: low density polyethylene (LDPE) and high density polyethylene (HDPE), polypropylene (PP), polyamide (PA), polyvinyl chloride (PVC), polyvinyl ester, polyester polyethylene terephthalate (PET), copolymers composed of at least two of these polymers, or mixtures of these polymers. Since the covering film remains on the skin during the use of the system, it should be as flexible and elastic as possible in order to ensure appropriate comfort when being worn by the patient. Inflexible films lead to the formation of folds and to the skin being cut into. LDPE and HDPE, in particular, form suitable flexible and elastic covering films.

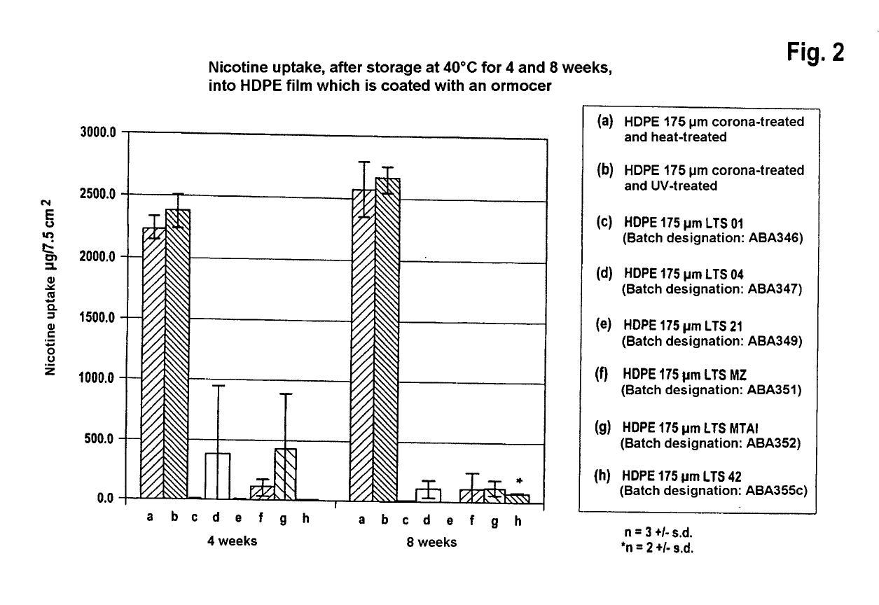

In order to investigate the suitability of ormocer-coated flexible covering films, an HDPE film of a thickness of 175 μm was coated with an ormocer lacquer of the abovementioned composition. The films, which were coated on both sides, were introduced into a gas space containing nicotine and the uptake of active compound by the films after 4 and 8 weeks of incubation at 40° C. was determined. The results of the investigations are shown in FIG. 2.

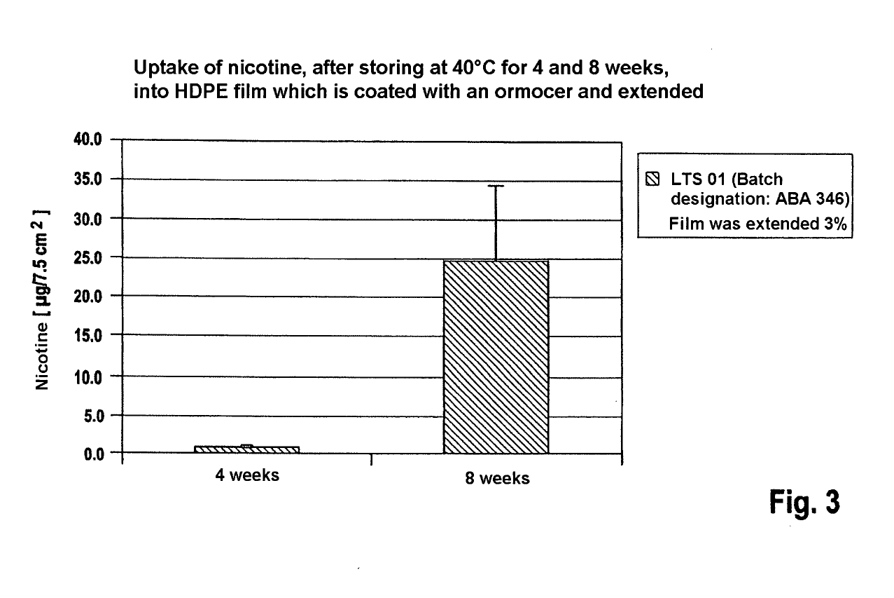

It was likewise possible to demonstrate that the covering films according to the invention possess good extensibility, that is consequently possess elasticity, and that the barrier properties are also preserved in the extended state. In order to show this, an HDPE film which was coated with the hybrid polymer ormocer was extended by 3% and the uptake of active compound after 4 and 8 weeks of exposure to nicotine was determined. The results are depicted in FIG. 3.

1. A dermal or transdermal therapeutic system comprising a peelable protective layer, a reservoir or matrix layer containing at least one active compound and a covering film having a barrier effect against gases, aromas and readily volatile substances, characterized in that the covering film is composed of at least one supporting layer and at least one layer composed of an ormocer.

2. The dermal or transdermal therapeutic system as claimed in claim 1, characterized in that the ormocer is obtained by the hydrolytic condensation of organically modified silicon dioxides.

3. The dermal or transdermal therapeutic system as claimed in claim 1, characterized in that the supporting layer is composed of at least one organic polymer.

4. The dermal or transdermal therapeutic system as claimed in claim 3, characterized in that the organic polymer is composed of low density or high density polyethylene, or of polypropylene, polyamide, polyvinyl chloride, polyvinyl ester or polyester, of a mixture of at least two of these polymers or of at least one copolymer composed of at least two monomers of the abovementioned polymers.

5. The dermal or transdermal therapeutic system as claimed in claim 1, characterized in that the supporting layer is composed of polyethylene terephthalate.

6. The dermal or transdermal therapeutic system as claimed in claim 1, characterized in that the ormocer layer has a thickness of between 1 μm and 10 μm.

7. The dermal or transdermal therapeutic system as claimed in claim 1, characterized in that the ormocer layer is applied on the supporting layer such that it is adjacent to the active compound-containing layer.

8. The dermal or transdermal therapeutic system as claimed in claim 1, characterized in that in each case at least one ormocer layer is applied on both surfaces of the supporting layer.

9. The dermal or transdermal therapeutic system as claimed in claim 1, characterized in that it comprises a readily volatile active compound.

10. The dermal or transdermal therapeutic system as claimed in claim 9, characterized in that the active compound is nicotine.
